(19) United States
(12) Patent Application Publication
(10) Pub. No.: US 2008/0020408 A1
(43) Pub. Date: Jan. 24, 2008

(54) Anti-Ube4a/Ufd2b Polyclonal Antibody and Its Use Thereof as Diagnostic and Prognostic Marker of 11Q23 Region Alterations

(76) Inventors: Spagnoli Luigi Giusto, Nerola (IT); Pucci Sabina, Roma (IT); Contino Gianmarco, Roma (IT); Citro Gennaro, Roma (IT); Novelli Giuseppe, Roma (IT)

Correspondence Address: PEARNE & GORDON LLP, 1801 EAST 9TH STREET, SUITE 1200, CLEVELAND, OH, 44114-3108, US

(21) Appl. No.: 10/589,299

(22) PCT Filed: Feb. 8, 2005
(86) PCT No.: PCT/IB05/00305

§ 371 Date: Jun. 21, 2007

(30) Foreign Application Priority Data
Feb. 16, 2004 (IT) MI2004A000251

(51) Int. Cl. G01N 33/574; C07K 16/18

(52) U.S. Cl. 435/7.23; 530/389.1

(57) ABSTRACT

The subject of this invention is an anti-UBE4A/Ufd2b polyclonal antibody. Moreover, this invention refers to the use of said antibody as a diagnostic and prognostic marker of alterations in the 11q23 region.

The subject of this invention is an anti-UBE4A/Ufd2b polyclonal antibody. Moreover, this invention refers to the use of said antibody as a diagnostic and prognostic marker of the alterations in the 11q23 region. The nature and the function of the antibodies are known.

Generally antibodies, also called immunoglobulins (Ig), are proteins produced by Vertebrates in response to the presence within the body of an antigen, i.e. foreign molecules, organisms or other agents. Antibodies are prevalently produced by plasma cells (terminal lymphocytes-B differentiation) and circulate in blood and lymphatic vessels, where meet and bind toantigens.

An antigen is then a substance that, once introduced in an organism, is able to induce an immune response and combine with antibodies and cellular receptors.

An antigen has got two properties: the immunogenicity, that is the property to induce a humoral (antibodies) or cellular immune response and the antigenicity, that is the ability to react in a selective way with specific antibodies. The part of the antigen that represents the binding site to the antibody is called epitope. Antibodies are able to chemically combine themselves, in a highly specific way, with the antigen molecule which has induced their production (epitope), in order to produce an antigen-antibody complex which triggers the inactivation of the antigen itself or its removal by means of macrophages phagocytosis. From a biological point of view, the antibody response represents one of the major mechanisms of an organism to protect itself from the action of foreign molecules and pathogen organisms.

It is also known that in biomedical research and diagnostic, as in the clinical practice, antibodies can be produced by different methods and used as reagents capable of binding a specific antigen. They are used, for example, to determine the exact sub-cellular localization of an antigen or to isolate it from a molecular complex, to detect interaction of the antigen with other macromolecules, determine its precise concentration or use it as a diagnostic tool for a neoplasia. Indeed, in a diagnostic perspective, the most common methods based on chemical-enzimatic reactions are now substituted by the use of antibodies that allow to visualize, on sections of tissue placed on a slide, the presence, both qualitatively and quantitatively of a particular antigen trough a known technique called immunohistochemistry. This technique allows to quickly obtain information which accurately reflect the discariocinetic (dependent on chromosomes) and DNA alterations which may result from the neoplasia. These are chromosomal alterations of the tumor cell whose epiphenomenon is the variation in the expression of some antigens. Some chromosomal regions are more frequently involved by translocations, deletions, amplifications, etc. Alterations of the 11q23 region, which are specific for some kind of high incidence tumours such as Neuroblastoma, Acute Lymphoid Leukaemia and Acute Mieloid Leukaemia, for example are know. Furthermore, the improvement of the techniques will allow to identify other tumours that have alterations in this region. Although it is possible for some neoplasias to obtain the diagnosis just with an histological test, (i.e the tissue analysis with optical microscope), the chance to get additional information about molecular alterations (DNA) of a tumor presents an advantage which cannot be renounced. In fact this allows to stratify the group of patients affected by the same neoplasia in subgroups with different prognosis and then decide for the best therapeutic protocols. Moreover, encouraging results show how the same molecular alteration can often represent the target of an aimed therapy, by allowing in the near future a radical revolution in the cancer pharmacological therapy. So it would be desirable being able to provide a specific antibody which is able to react selectively with a specific antigen to diagnosticate molecular alterations which, for example in tumours involving 11q23 region, allow us to understand the tumour biology, define the prognosis and give us therapeutic indications.

In the biomedical practice, the most frequent target of the antibodies production of the produced antigens are the proteins that represent the structural and functional substrate of all biological processes.

One protein is represented, in its primary structure, by an amino acid sequence. Amino acids are assembled in a specific order, codified by the corresponding gene in the cellular DNA. A large number of different proteins are required to realize the complex structures and the biological processes needed by the cell. Within the same organism all the somatic cells contain the same informations at the DNA level, and, in theory, they are able to assemble all the proteins. However this cells express the several proteins in a different way through complex regulation systems according to the tissue of which they are part and the specific function they carry out (for example a neuron and a muscular cell). First of all, there is therefore the need of having a tool to characterize the tissue-specific expression patterns of several proteins. Trough a comparison of these expression patterns we may detect alterations in pathological tissues. Once we are able to selectively identify a protein we can detect its interactions with other proteins and macromolecules.

The understanding of these interactions is an essential step to identify the cellular biology and the complexity of its functions. In fact one cell must answer dynamically to the modifications of the micro-environment by modulating the expression levels of the single proteins and degrading them when their function in no more necessary. For example one can think of the cellular cycle control factors, of the proteins needed for the proteinic syntesis, of the DNA repair systems, and of the Chaperonine and Heat Shock Protein system, that drive proteins to recover their original structure following on stress events. Of a particular interest is on this matter the study of the ubiquitination pathway. Ubiquitination is a known fundamental cellular process that consist in linking a protein called ubiquitin to a protein that must be degraded. The ubiquitin represents the degradation signal for the proteosome 26S (ATP-dependent protease) that is responsible of the degradation of proteins whose levels are regulated in response to modifications in the cellular environment. This mechanism guarantees a protein turn-over essential for the cell survival and mediates the degradation of proteins responsible for activities such as cell cycle progression, stress response, antigens processing, cellular signaling, DNA repair, apoptosis. It is not surprising that alterations of this pathway have been variously implicated in neurodegenerative diseases (like Alzheimer and Parkinson's), cumulatives and in neoplastic events wherein the cell gains an autonomous growth loosing the control on the cellular cycle and without being able to undergo the apoptosis mechanism. Therefore it remains the need to provide a specific antibody, which is selective for a specific antigen and able to diagnose molecular alterations involved in the 11q23 chromosomal region by allowing to understand the tumour biology, to define the prognosis and give therapeutic indications.

A first aim of the present invention is to provide an antibody able to detect the presence of a certain antigen both at qualitative and quantitative level through the immunohistochemical technique.

Another object of the present invention is to provide a diagnostic and prognostic method to determine the alterations that the tumoral cell has underwent at the chromosomal level in the 11q23 region.

A further aim of the present invention, is to supply an antibody able to selectively react with a specific antigen, in order to diagnose molecular alterations in cancers that involve the 11q23 region such as in the case of Neuroblastoma, Acute Lymphoid Leukaemia and Acute Mieloid Leukaemia. Yet another aim is to supply a polyclonal antibody able to selectively identify a protein involved in the ubiquitination pathway.

The subject of the present invention is an antibody characterised by the properties listed in the principal appended claim.

Another object of the present invention is the use of said antibody in the field of research, diagnostic and medicine as indicated in the principal appended claims.

Another object of this invention is a diagnostic and prognostic kit as a marker of molecular tumoral alterations with the characteristics listed in the appended claim.

The Applicant has carried out research studies in order to develop and produce an antibody specific for the antigen formed by the human and murine protein UBE4A/Ufd2b.

The Applicant has focused its studies on human and murine protein UBE4A because the region of the genome in which UBE4A is located (11q23 chromosome) is known to be deleted in some tumoral forms.

UBE4A is a protein involved in the Ubiquitination process.

In particular UBE4A/Ufd2b is an “E3 ligase”. E3 ligases form part of a numerous proteins family that, in the ubiquitination process, are responsible for the specific selection of the protein to be degraded. It is known that alterations of one of these proteins result in the accumulation or in the excessive function of proteins that should be degraded, with a consequent cellular, of an organ or systemic damage. Moreover UBE4A/Ufd2b can also act as a polyubiquitination factor, i.e. it elongates the ubiquitins chain linked to the protein that must be degraded, increasing the degradation efficiency. UBE4A/Ufd2b functional domain is called U-box and is represented by a portion of about 64 aa situated in the C-terminal part of the protein. This domain is highly conserved through the evolution and, in other organisms, interactions with proteins involved in the cell cycle progression and in the assembly both at the mRNA level and in the folding of proteins have been shown.

The Applicant, in order to produce the specific antibody for the antigen formed by the UBE4A/Ufd2b protein has used the known immunization technique. In practice, the immunization is a known process for the production of an antibody able to bind a specific protein.

The Immunization consists of immunogenizing a rabbit or another animal with a specific epitope which is characteristic of the target protein. Different plasma cells clones will produce antibodies able to recognize the selected epitope. These antibodies will be present in big amount in the animal serum that represents then a good source of polyclonal antibodies. The epitope is represented by a portion of the protein, that is an amino acids sequence having a length 10 to 25 amino acids (aa).

The Applicant has analyzed the UBE4A/Ufd2b human (homo sapiens) protein amino acid sequence and has compared said sequence with the mouse (mus musculus) amino acidic sequence. The identification of the antigenic sites of the two sequences has been done using an algorithm developed by Kolaskar A. S. and Tongaonkar P. C. (1990) which is available as a software called “Antigenic Peptide Prediction (www.mifoundation.org)”.

The Applicant has then located the sequences having an antigenic site shared by homo sapiens and mouse and has focused his attention to the N-terminal sequences. The choice of an antigenic site sequence must preferably regards the N and C-terminal regions (respectively the first and the last aa of the sequence). These regions are usually accessible to the solvents and are not folded even in the native form (not denatured). Moreover antibodies directed against these parts of the sequence even recognize the native proteins. C-terminal antigenic sites have been excluded for three foundamental considerations: (a) because they involve the region of the protein (U-box) where the single functional domain is located and for this reason are subject to a greater preservation at an evolutionistic level in other species and other proteins which, by containing the U-box domain, are different from the UBE4A/Ufd2b protein; (b) because C-terminal antigenic sites of the U-box are binding sites for the substrate and then the binding with the antibody wouldnt allow the binding of the specific substrate of the protein, by making studies for identifying the interaction means of UBE4A impossible; (c) because being placed into a domain, or close to it, the protein is likely to be modelled according to its secondary and tertiary structures, modifying the accessibility of the epitope to the antibody.

The consideration expressed in point (c) is also the reason of the exclusion of the central regions of the sequence. In fact, in the attempt of identifying proteins in a proteinic extract from tissue (western blot) is technically possible to linearize the protein amino acid sequence by denaturing it. This operation is not possible on tissue sections (immunoistochemistry) where proteins maintain their native structure or, alternatively, in the case of studying interaction with other proteins (immunoprecipitation). Sometimes antibodies, even if commercialized, are not able to work on tissues, leading to a remarkable disadvantage on the level of technical applications. Moreover the sequence must be specific for this protein, that is not present in sequences of other human and murine proteins.

Advantageously, the Applicant has selected among a group of interesting sequences, a small amino acid sequence chosen into the N-terminal region according to the rules explained so far. The choice of the shortest sequence guarantees the greatest specificity and efficiency of the antibody against the antigen and, at the same time, the lowest presence of cross-reactions, that is the non specific binding of the antibody with proteins other than UBE4A/Ufd2b.

The amino acid sequence, that constitutes the object of this invention, has been identified and selected as follows:

Ile-Ser-Ser-Asn-Pro-Phe-Ala-Ala-Leu-Phe-Gly-Ser-Leu-Ala-Asp-Ala-Lys-Gln (ISSNPFAALFGSLADAKQ). Said lowest amino acid sequence, which is the object of the invention, corresponds to the sequence of aminoacids from 10 to 27 of both human and murine UBE4A protein.

Clearly, the invention shouldn't be limited to this sequence but also to the sequences that can be derived from it.

The amino acid sequence (or peptide) was synthesized in vitro by using methodologies and techniques known by those skilled in the art.

The above amino acid sequence constitutes the epitope of the protein UBE4A/Ufd2b (antigen), for which the rabbit has produced the antibody.

It is known that small molecules, such as peptides, although they are able to interact with the products of an immune response (such as the antibodies), may not be able to elicit it. Generally, in order to render them completely immunogenic, they are conjugated with a carrier protein

In this case we used the Bovine Serum Albumine (BSA), commonly used for its stability and solubility in plasma and for the ability of imparting immunogenicity to the conjugated peptide. For the conjugation of the peptide with BSA the “Imject® Immunogen EDC Conjugation kit” by PIERCE (Rockford, Ill., US) was used according to the producer instructions

After having obtained a solution containing the conjugate peptide-protein carrier, we injected 150 μl by subcutaneous injections on the back of the rabbit. Seven days after the first administration, the operation has been repeated in the same animal in order to support and increase the specificity of the immune response. The operation has been then repeated every 7 days for 6 times.

The immune response has been checked starting form the third immunization cycle, using the known ELISA technique which is able to measure the antibody response on rabbit serum. Rabbit serum has been obtained from a blood sample of 5 ml from auricular vein of the rabbit. As a “blank” for the ELISA test, the serum of the rabbit taken before the first immunization has been used (pre-immune serum).

The polyclonal antibody found after six immunization cycles allowed to analyze the expression profiles of the UBE4A protein on a series of both murine and human tissues.

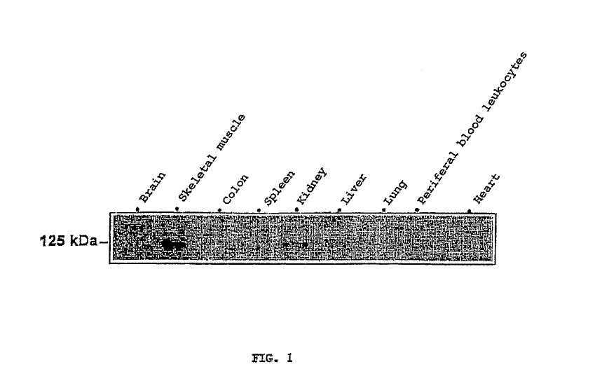

The study of the expression on proteic extracts-containing lysates from human and murine tissues allowed us to analyze the expression of the protein both qualitatively and quantitatively. In fact by means of the known Western Blotting technique the antibody is able to recognize the UBE4A Ufd2b protein as a unique band of about 125 kDa (FIG. 1) and 130 kDa in humans and mouse respectively. This data demonstrates that, from one side, the antibody didn't show cross-reactions with other proteins and it is then highly specific and from the other side that the protein doesn't show isoforms or post-transcriptional modifications, as the molecular weight is approximately the molecular weight obtained from the addition of the molecular weight of the single amino acids.

Thereafter, the applicant has tested the efficiency of the antibody on sections of human tissue by the known technique called immunohistochemistry.

The immunohistochemistry determines the presence of an antigen in healthy or pathological tissues. Many antibodies “work” very well with different methods but they can be completely infective on tissue.

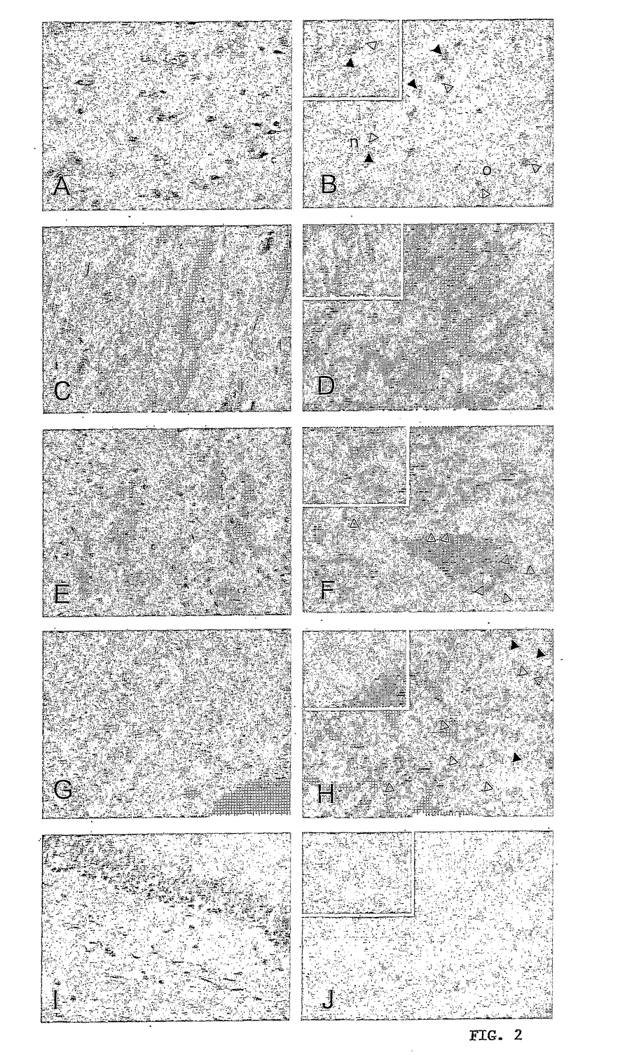

For example, in the case of brain a section of 5 μm of tissue in paraffin then mounted on positive charged slide. Subsequently, the section has been deparaffinized and treated with xylene and alcohol. Then, the sections have been treated with the polyclonal antibody anti-UBE4A, which is the object of the invention (primary antibody) with a 1:2000 dilution for 1 h at room temperature. Subsequently, some washes in order to eliminate the excess of antibody have been carried out and the sections incubated with anti-IgG serum of the biotinilated rabbit (secondary antibody) and then with streptavidin-bound oxidase The staining was completed by incubating the sections with a chromogen solution. At this point, after a wash, the hematoxylin dye has been used in order to give a contrast to the cellular structures. The section has then been covered with a slide and analysed under the optical microscope (FIG. 2). Immunohistochemistry on brain tissues allowed us to localize UBE4A protein in nucleus and cytoplasm of cortical neurons and oligodendrocytes where the Ubiquitination plays a major role in regulating differentiative switches.

In contrast, the himmunoistochemistry on sections in paraffin embedded renal tubules showed a typically cytoplasmic expression.

Staining on liver sections showed a diffusely cytoplasmic pattern and a particular nuclear expression: in fact, the hepatocytes on a same section appeared to be positive, weakly positive or negative at the immunohistochemistry, suggesting that UBE4A/Ufd2b has a cell cycle dependent expression or plays a role in the cell cycle control per se. Further, the presence of cytoplasmic staining in neurons and hepatocytes suggests that UBE4A/Ufd2b contributes to the degradation of specific substrates related to neuronal and hepatic functions (FIG. 2).

UBE4A/Ufd2b is also one of few genes that map within the 2-5 cM of the 11q region which frequently results deleted in cases of neuroblastoma without amplifications of N-Myc and with an unfavourable prognosis.

Krona et al. have recently reported that the human homologous of UBE4A, that is UBE4B which maps on the chromosome 1p36, is deleted in all the cases of their survey of NBL of 3^{rd} stage and mutated in a case of NBL.

Moreover, rearrangements in the 11q23 region are seen in many cases of acute lymphoid leukaemia and acute myeloid leukaemia. Although in these leukaemias, the translocations against the chromosome 11 mainly concern the MLL gene, which maps close to UBE4A/Ufd2b, cases were described in the literature, wherein in the translocation 11q23 may involve different genes not identified but other than MLL

The antibody of this invention, also allowed us to detect a misregulation in the expression of UBE4A/Ufd2b tested on a panel of malignant human tissues by immunohistochemistry. UBE4A/Ufd2b is over-expressed in the desmoplastic reaction, i.e. the productive reaction to the neoplasia which express itself with the genesis of a stroma for the epithelial neoplasia.

FIG. 1 shows the expression of UBE4A/Ufd2b by Western Blotting. The analysis on protein extracts from human tissues shows the UBE4A/Ufd2b protein, identified by the anti-UBE4A polyclonal antibody as a single band of about 125 kDa. UBE4A/Ufd2b is expressed at high levels in the skeletal muscle, kidney and liver and faintly in the peripheral blood leukocytes and spleen.

The figure shows a study of the expression through Immunohistochemistry of human UBE4A/Ufd2b protein in the cerebrum (A, B), heart (C, D), kidney (E, F), liver (G, H) and lung (I, J). Sections of human tissue were stained on the left with ematoxiline-eosine (A, C, E, G and I) and on the right with the polyclonal antibody for the UBE4A/Ufd2b protein (B, D, F, H and J). The specificity of the immunohistochemical reaction was confirmed through the comparison with sections stained with the pre-immune serum of the rabbit used at the same dilution of the primary antibody (data not shown). The positivity of the staining with the anti-UBE4A polyclonal antibody in the nucleus is indicated by the “empty triangle” arrows, staining in the cytoplasm by the “dark triangle” arrows. As it is shown, UBE4A/Ufd2b was expressed predominantly both in the nucleus and the cytoplasm of cortical neurons (B; n) and oligodendrocytes (B; o), in the nucleus of renal tubule cells (F). In the liver, UBE4A/Ufd2b showed a nuclear expression and a weak citoplasmic expression (H) in most hepatocytes

The expression of UBE4A/Ufd2b was absent in the heart and lung (D and J).

1. Polyclonal antibody anti-UBE4A/Ufd2b.

2. Antibody according to claim 1, characterized by being specific for the human and murine UBE4A protein antigen in the portion relative to the sequence of the epitope at the 10-27 position.

3. Antibody according to claim 2, characterized in that said sequence, corresponding to amino acids 10 to 27 in the sequence of the human and murine UBE4A protein is composed as follows: lle-Ser-Ser-Asn-Pro-Phe-Ala-Ala-Leu-Phe-Gly-Ser-Leu-Ala-Asp-Ala-Lys-Gln.

4. Antibody according to claim 3, characterized in that it is conjugated with bovine serum albumine (BSA) as a carrier protein.

5. Immunohistochemical method to determine UBE4A/Ufd2b protein antigen using the antibody according to claim 1.

6. Diagnostic and prognostic method to determine molecular abnormalities in the region 11q23 characterized in that it includes a step wherein the antibody according to claim 1 selectively reacts with the UBE4A/Ufd2b protein antigen.

7. Method according to claim 6, characterized in that said molecular alterations are consequent to tumours: Neuroblastoma, Acute Lymphoid Leukaemia, Acute Myeloid Leukaemia.

8. Method to evidence any misregulation of the UBE4A/Ufd2b expression in samples of neoplastic human tissues affected by neurodegenerative diseases using Immunohistochemistry.

9. Diagnostic and prognostic kit to be used as a marker of the alterations of the region 11q23, characterized in that it includes the antibody according to claim 1.
